(19) United States
(12) Patent Application Publication
(10) Pub. No.: US 2002/0147402 A1
(43) Pub. Date: Oct. 10, 2002

(54) Method for diastolic blood pressure measurement

(75) Inventors: Meir Nitzan, Belt El (IL); Chaim Rosenfeld, Jerusalem (IL)

Correspondence Address: DR. MARK FRIEDMAN LTD., C/O BILL POLKINGHORN, DISCOVERY DISPATCH, 9003 FLORIN WAY, UPPER MARLBORO, MD, 20772, US

(73) Assignee: NINBAR Ltd.

(21) Appl. No.: 10/117,999

(22) Filed: Apr. 9, 2002

Related U.S. Application Data
(60) Provisional application No. 60/282,180, filed on Apr. 9, 2001.

(51) Int. Cl. A61B 5/02

(52) U.S. Cl. 600/494; 600/485

(57) ABSTRACT

A method for measuring arterial diastolic blood pressure in a subject includes deriving values of a delay between pulses in two signals indicative of cardiac induced pulsatile variations of a cardiovascular parameter in a first region and a second region of the subject's body. A variable pressure applied to a region of the subject's body causes variation of the delay. A difference curve is calculated by subtracting from the delay values a monotonic mathematical function adjusted as a best-fit to the data. A value of the variable pressure for which the difference curve exhibits a stationary point is then identified as the diastolic blood pressure.

### FIELD AND BACKGROUND OF THE INVENTION

This invention relates to a refinement of a method and corresponding device for measurement of blood pressure, and particularly diastolic blood pressure (DBP), which is described in U.S. Pat. No. 6,120,459 and the related PCT Patent Publication No. WO0074563, both of which are hereby incorporated by reference in their entirety as if fully laid out herein.

According to a preferred embodiment of the aforementioned U.S. patent, DBP is derived from the measurement of the time-delay (TD) of a PPG pulse in the finger distal to a pressure cuff wrapped around the arm, relative to the PPG pulse in a contralateral finger. TD is measured as a function of the cuff pressure P_{C}, and the cuff pressure for which TD has a predefined value is taken to be the DBP. The preferred value for this predefined value of TD was suggested to lie in the range between 15 and 25 ms.

In the aforementioned PCT application, it was noted that the predefined value of TD is not constant for all patients, but depends on the systolic blood pressure (SBP) value for a given patient. Thus FIG. 21 in the PCT Application shows data of the time-delay at DBP (TD(DP)) as a function of SBP and the corresponding regression curve (where the DBP was measured by sphygmomanometry (SPM)). According to the teachings of the PCT application, the DBP in a specific examination is obtained as follows: Firstly the SBP is obtained from the reappearance of the PPG pulse when the cuff pressure decreases below SBP value. Then the required said predefined value of TD is obtained from the aforementioned regression curve representing TD(DP) vs. SBP data. The DBP is then identified as the value of P_{C} which corresponds to the value of TD in the TD vs. P_{C} curve for the SBP value of that individual. This method is more accurate than that described in the US patent, but is still inaccurate, since the curve of TD(DP) vs. SBP—as shown in FIG. 21 (PCT)—is only a rough approximation to the true dependence of TD(DP) vs. SBP, which exhibits significant scatter of data points around the said regression curve.

### SUMMARY OF THE INVENTION

Without limiting the present invention to any specific physiological model, it is believed that the time delay measured between PPG signals in the contralateral fingers is a sum of the effects of at least two different phenomena, one of which falls to zero at cuff pressures less than DBP and another of which continues to produce a time delay even below the DBP. For this reason, it has been observed that the variation of TD with cuff pressure P_{C} exhibits a change of (the generally negative) gradient from more moderate to (negative) steeper when P_{C} is in the vicinity of the DBP.

In principle, the desired value may be derived directly from the graph of TD against P_{C}. In practice, it has been found that superior results are obtained by instead identifying a maximum (or minimum, depending upon sign conventions) value of a difference between the measured TD and a monotonically reducing mathematical function fitted to the data.

Thus, according to the teachings of the present invention there is provided, a method for measuring arterial diastolic blood pressure in a subject, the method comprising: (a) generating first and second signals indicative, respectively, of cardiac induced pulsatile variations of a cardiovascular parameter in a first region and a second region of the subject's body; (b) processing the first and second signals to derive values of a delay between pulses in the first signal and corresponding pulses in the second signal; (c) applying a variable pressure to a pressure application region of the subject's body so as to affect blood flow through at least one artery in the pressure application region, the variable pressure being varied over time, the first region, the second region and the pressure application region being chosen such that the delay varies as a result of changes in the variable pressure; (d) deriving parameters of a mathematical function such that the function corresponds approximately to a relationship between the delay and the variable pressure, the mathematical function being monotonic; (e) calculating a difference between the values of the delay and corresponding values on the mathematical function; and (f) identifying as the diastolic blood pressure a value of the variable pressure for which the difference exhibits a stationary point.

According to a further feature of the present invention, the mathematical function is an exponential function.

According to a further feature of the present invention, the mathematical function is a power-curve function.

According to a further feature of the present invention, the mathematical function is a polynomial function.

According to a further feature of the present invention, prior to the step of identifying, an approximate value of the diastolic blood pressure is obtained, and the identifying includes selecting a stationary point of the difference from a plurality of stationary points by proximity to the approximate value.

According to a further feature of the present invention, the approximate value is obtained by identifying a value of the applied pressure at which the delay assumes a predefined non-zero value.

According to a further feature of the present invention, the predefined non-zero value of the delay is calculated from a predefined function of the systolic blood pressure of the subject.

According to a further feature of the present invention, the parameters are derived according to a best-fit criterion. Preferably, a least-squares best-fit criterion is used.

According to a further feature of the present invention, both the first and the second signals are generated by PPG sensors.

According to a further feature of the present invention, at least one of the first and the second signals is generated by a PPG sensor.

According to a further feature of the present invention, one of the first and the second signals corresponds to oscillations in air pressure within a cuff used to apply the variable pressure.

### BRIEF DESCRIPTION OF THE DRAWINGS

The invention is herein described, by way of example only, with reference to the accompanying drawings, wherein:

FIG. 1. (a—above) The systolic increase in the arterial blood pressure (ABP) pulse. When the cuff air pressure Pc is applied to the arm, the arteries are closed for ABP<P_{C}, and the flow through the artery starts only after a time-delay TD.

(b—below) The theoretical curve of the time-delay TD as a function of the cuff air pressure P_{C}. The curve in (b) is the curve in the insert in (a), rotated by 90°.

FIG. 2. Schematic drawing of the blood pressure measurement system: 1. Pressure cuff. 2. PPG probes, in a finger distal to the pressure cuff, and in a finger in the contralateral hand (not seen in the figure). 3. Mercury manometer. 4. Piezoelectric transducer. 5. Pressure pump and its electronic control. 6. Electronic control of the PPG probes. 7. Computer with A/D card.

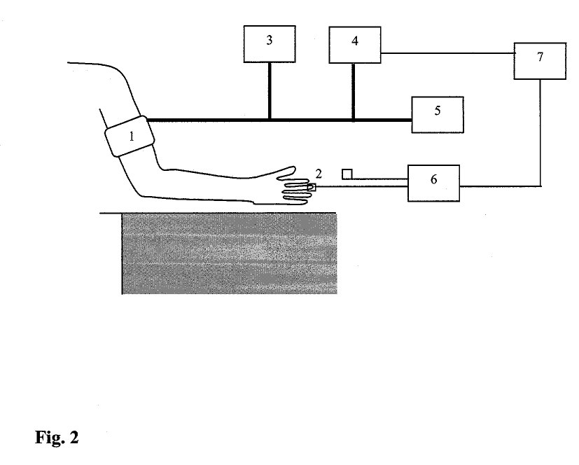

FIG. 3. The PPG pulses in the two hands at various values of cuff air pressure P_{C} for one of the subjects. Note that the time-delay TD between the start time of the two PPG pulses increases with P_{C}. TD is greater than zero for cuff pressure value which is equal to the value of DBP (80 mmHg).

FIG. 4. The value of TD for cuff pressure P_{C} which is equal to DBP, TD(DP), as a function of SBP for 60 subjects (180 examinations). The regression curve of third degree polynomial is also shown.

FIG. 5. The curves of the PPG and the cuff pressure as a function of time for one of the subjects. The PPG pulses reappear when the cuff pressure decreases to below systolic blood pressure. The systolic blood pressure as obtained by sphygmomanometry (SBP_{S}) and the corresponding time are marked by dashed lines.

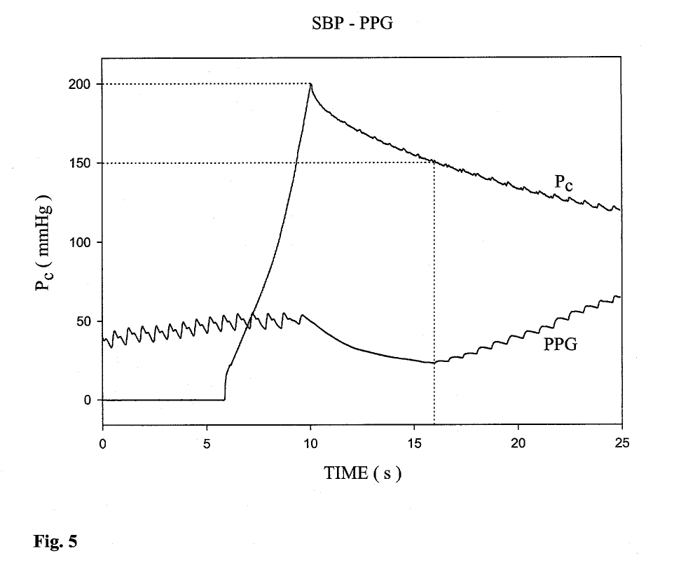

FIGS. 6. The time-delay TD, the best-fit 4^{th} order polynomial curve and their difference, ΔTD, as a function of the cuff pressure during the deflation period, for two examinations. The value of DBP is also shown.

FIG. 7A and 7B The time-delay TD, the best-fit exponential curve and their difference, ATD as a function of the cuff pressure during the deflation period, for two examinations. The value of DBP is also shown.

FIG. 8. The values of DBP_{P} as a function of DBP_{S}. The regression line and the x=y line are also shown.

FIG. 9. The values of SBP_{P} as a function of SBP_{S}. The regression line and the x=y line are also shown.

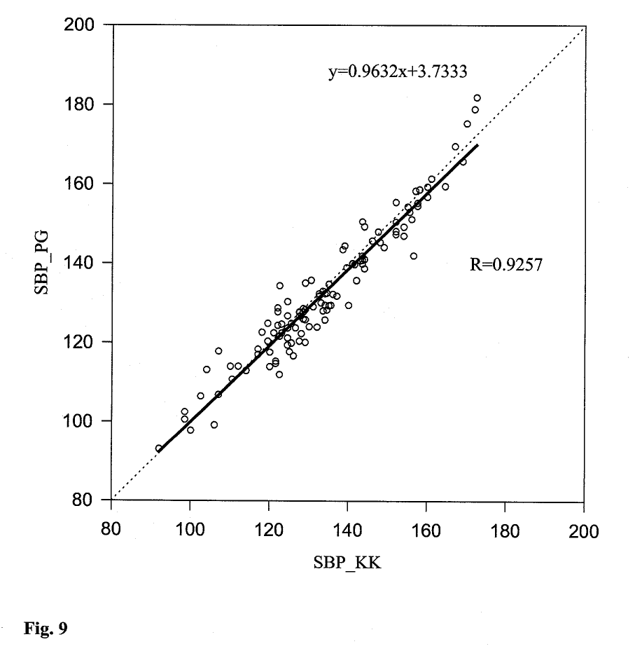

### DESCRIPTION OF THE PREFERRED EMBODIMENTS

The present invention is a method for measuring arterial diastolic blood pressure in a subject.

The principles and operation of methods according to the present invention may be better understood with reference to the drawings and the accompanying description.

In general terms, the method of the present invention includes (a) generating first and second signals indicative, respectively, of cardiac induced pulsatile variations of a cardiovascular parameter in a first region and a second region of the subject's body; (b) processing the first and second signals to derive values of a delay between pulses in the first signal and corresponding pulses in the second signal; (c) applying a variable pressure to a pressure application region of the subject's body so as to affect blood flow through at least one artery in the pressure application region, the variable pressure being varied over time, the first region, the second region and the pressure application region being chosen such that the delay varies as a result of changes in the variable pressure; (d) deriving parameters of a mathematical function such that the function corresponds approximately to a relationship between the delay and the variable pressure, the mathematical function being monotonic; (e) calculating a difference between the values of the delay and corresponding values on the mathematical function; and (f) identifying as the diastolic blood pressure a value of the variable pressure for which the difference exhibits a stationary point.

For the purpose of the present application, the term “monotonic” is used to refer to a mathematical function for which, given the parameters chosen, the gradient does not change sign (i.e., cross zero) over the range of measured values. The behavior of the function outside the region fitted to the measured data is not considered.

In a further matter of terminology, it should be noted that the value of DBP is taken as a stationary point on the curve of the difference. According to the sign conventions for calculation of the difference as illustrated herein, the value will always be identified at a maximum of the difference. Clearly, by changing sign conventions, the difference may be inverted such that the value of interest is a minimum.

Various monotonic functions, or locally monotonic functions, may be used to implement the present invention. In certain cases, such as by use of polynomials, it is preferable to supplement the basic method with an intermediate step of obtaining an approximate value of the diastolic blood pressure. This approximate value is then used to help identify which stationary point should be used to yield the accurate value of DBP. While any technique may be used to obtain the approximate value, most preferred implementations of the invention use the techniques described in the above-referenced coassigned US and PCT publications.

In particularly preferred implementations where the mathematical function is an exponential function or a power-curve function, the difference curve obtained typically has a single primary peak which is easily identified and which gives the desired precise DBP value without requiring the aforementioned intermediate step of obtaining an approximate value.

Typically, at least one signal is generated by a PPG sensor applied to a finger distal to the pressure cuff. The reference signal may be generated by a PPG sensor on a contralateral finger. Alternatively, the system may be structurally simplified by employing variations in air pressure within the cuff as a reference signal.

The invention will now be discussed further in the form of a possible physiological model for the invention and specific examples of an implementation. It should be appreciated, however, that none of these details should be interpreted as limiting the scope of the appended claims other than where they are expressly recited therein.

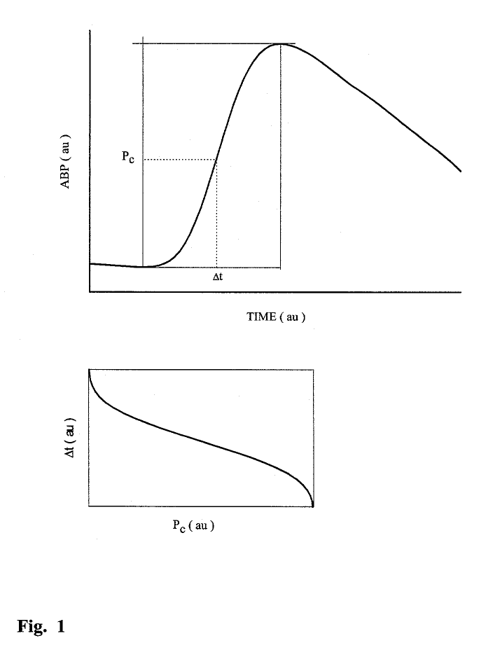

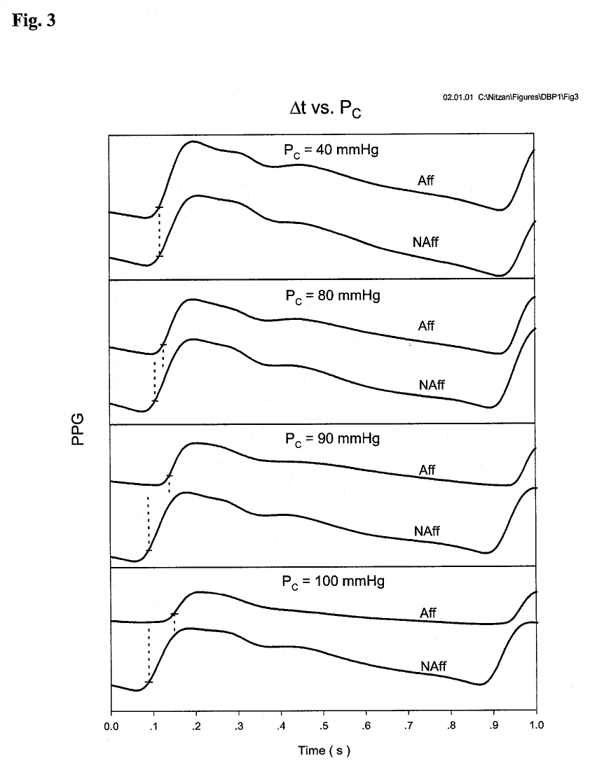

For the purpose of facilitating understanding of the invention, but without limiting the scope of the invention to any specific physiological model, it is believed that the mechanisms underlying the method of the present invention may be as follows. When the cuff pressure P_{C} is between DBP and SBP the artery under the cuff is closed for arterial blood pressure (ABP) below P_{C} and the pressure pulse cannot propagate distal to the pressure cuff. The artery opens only during that part of the cardiac cycle for which ABP increases above P_{C} so that the increase section of the pressure pulse distal to the pressure cuff starts with a time-delay TD (FIG. 1A). Hence, the start of the systolic increase in the PPG pulse is delayed in the sensor distal to the cuff relative to that in a sensor on the contralateral hand. Theoretically, this would result in a corresponding wave-like variation in TD with the cuff pressure P_{C} as shown in FIG. 1B. In practice, however, the relation is less simple, as will be seen from FIG. 3.

FIG. 3 depicts PPG pulses in the two hands at various values of cuff air pressure P_{C}, for one of the subjects having DBP value of 80 mmHg. It can be seen that the time-delay TD between the start time of the two PPG pulses increases significantly with P_{C} as expected from the model of brachial artery closure when ABP is lower than P_{C}. However, TD was found to be greater than zero even for cuff pressure values which are equal to or even somewhat smaller than DBP, indicating that additional phenomenon affects TD as well. This phenomenon is probably the lower transmural pressure (the difference between the internal blood pressure in the artery and the external pressure), for higher values of P_{C}. Lower values of transmural pressure are associated with higher arterial compliance, leading to lower pulse wave velocity and higher pulse transit time. Hence,. the start of the systolic increase in the PPG pulse is delayed in the sensor distal to the cuff relative to that in a sensor in the contralateral hand, and for higher values of P_{C} the time-delay TD will be higher. Thus the component of TD resulting from closure of artery above DBP appears as a small positive wave in the experimental TD vs. P_{C} curve, among other fluctuations. The detection of the DBP, corresponding to one end of this wave on the experimental TD vs. P_{C}, curve is preferably performed in two stages. One preferred, but non-exclusive, example of an implementation of these stages will now be described in detail.

1. Preprocessing and Optional Rough Approximation of DBP.

For each subject the time-delay TD in each pulse was measured as a function of P_{C} for P_{C} below SBP value. The TD vs. P_{C} curve was smoothed by means of moving average of 3 points (1+1+1 points), and the best-fit regression curve of a forth degree polynomial was drawn. (Other best-fit curves such as those of an exponential function (aexp(bx)) or power function (ax^{b}) can also be used).

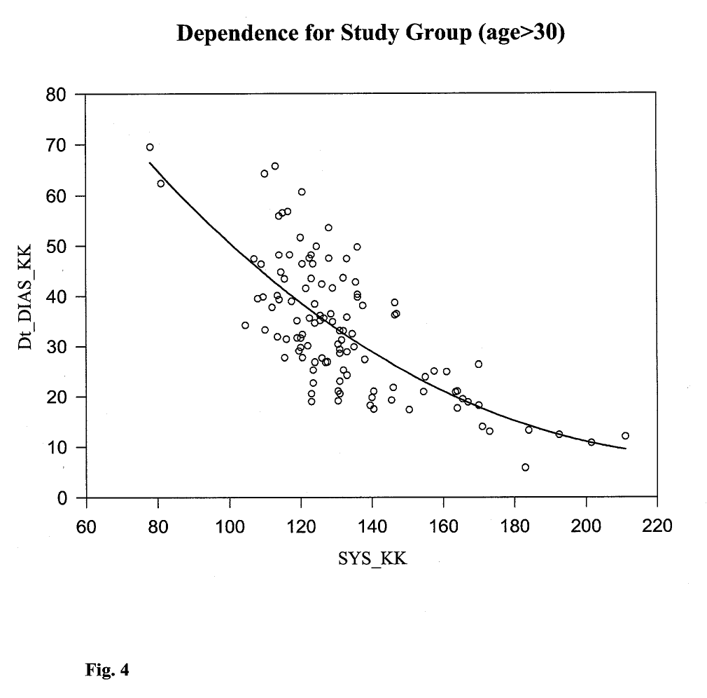

In certain cases, particularly where polynomial functions are used, it is helpful to perform an intermediate step of obtaining an approximate DBP value. This value is used for subsequent identification of the correct stationary point for precise determination, as will be detailed further. The value of TD for the value of P_{C} which is equal to DBP (TD(DP)) (obtained from the best-fit curve was found to depend on the SBP value, as shown in FIG. 4, which presents data, based on best-fit 4^{th} degree polynomial, from 180 examinations performed on 60 subjects (study group). The best-fit regression curve of a third degree polynomial was also drawn for the data of FIG. 4, and this curve is the basis for the derivation of DBP from the TD vs. P_{C} curve in another group of 61 subjects (working group). For each subject the value of SBP was obtained from the PPG pulses by the method described above, and the expected value of TD(DP) (i.e. the value of TD for P_{C}=DBP) was derived from the best-fit polynomial in FIG. 4. Then by inserting the value of TD(DP) in the individual TD vs. P_{C} curve, an approximation of DBP was obtained and named DBP₀. DBP₀ is only an approximation for DBP, since the individual TD(DP) values are scattered around the best-fit regression curve (as can be seen in FIG. 4), so that accurate determination of TD(DP ) from the value of SBP is not expected.

2. Refinement of DBP Measurement

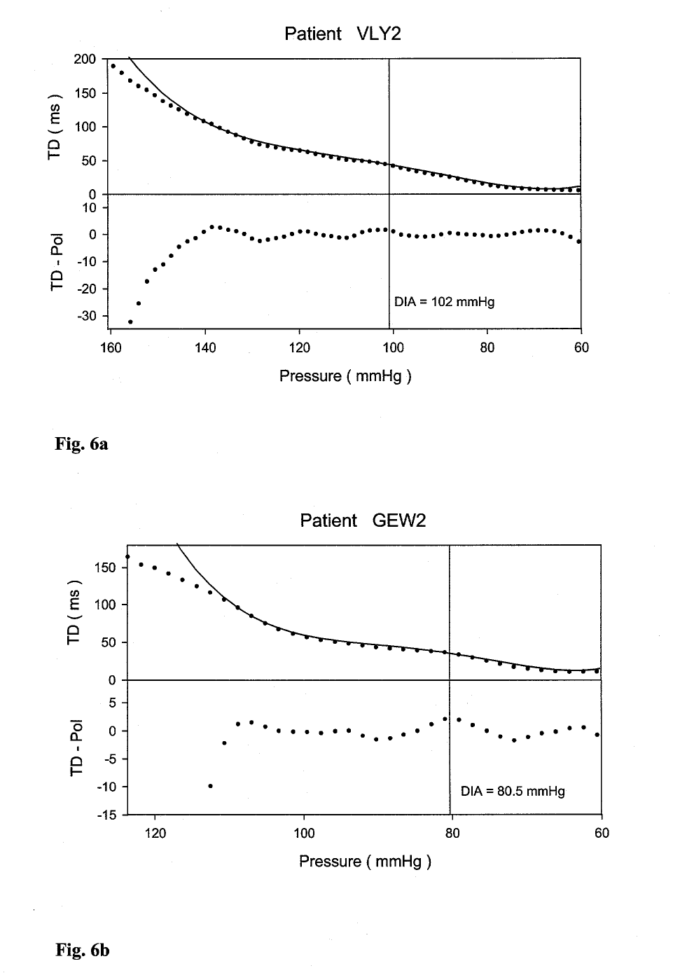

The required wave on the experimental TD vs. P_{C} curve was determined from the curve of ΔTD (i.e., the difference between TD values and the corresponding best-fit approximation—polynomial, exponential, power or other) as a function of P_{C}. FIGS. 6A and 6B present two examples of the experimental TD vs. P_{C} curve and the positive wave in the vicinity of DBP (above) and the corresponding ΔTD vs. P_{C} curve (based on polynomial best-fit approximation). It can be seen that several positive waves appear on the curves, so that the required wave has to be selected. On the ΔTD vs. P_{C} curve, “appropriate” positive wave of ΔTD was determined as a group of at least three consecutive PPG pulses, for which ΔTD was positive, and on the right side of the group (the lower P_{C} values) there are at least two consecutive pulses of negative ΔTD. The value of P_{C}, which is associated with the pulse of maximum value of ΔTD, was taken as DBP. If several appropriate waves were found, the wave nearest to DBP₀ was taken. Preferably a given positive wave is not considered “appropriate” if the maximum value of ΔTD for that wave is associated with a pulse of TD value above 60 ms or below 15 ms, or if the pulse of maximum value of ΔTD was not in the neighborhood of DBP₀ (i.e. for P_{C} between DBP₀−10 mmHg and DBP₀+15 mmHg). If no “appropriate” wave was found, the value of DBP₀ was taken as DBP.

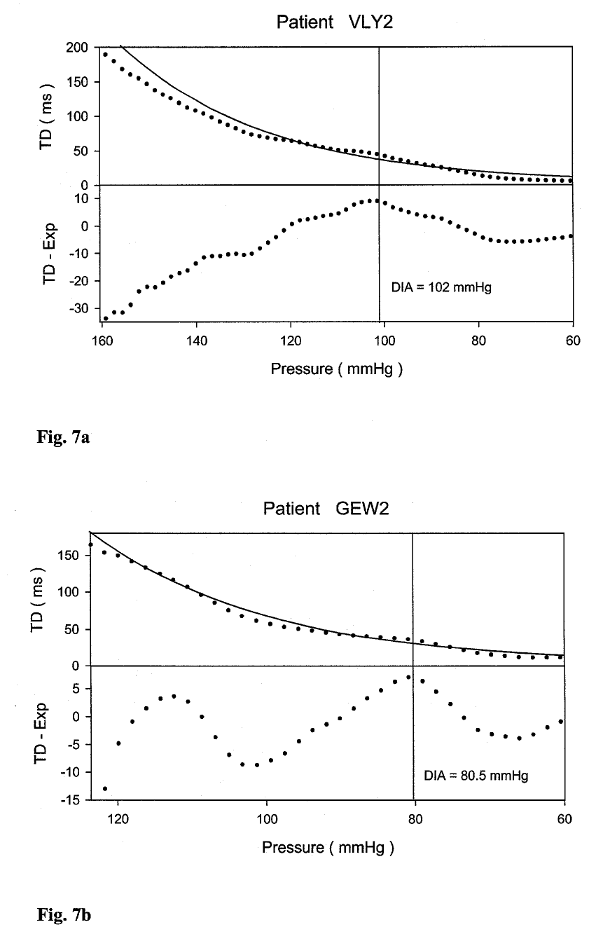

The above stated conditions for an “appropriate” wave are for polynomial approximation and they are different if the best-fit regression curve is that of an exponential curve (aexp(bx)) or power curve (ax^{b}). Furthermore, in most cases only a single wave was found on the experimental curve of ΔTD (i.e., the difference between TD values and the corresponding best-fit exponential or power approximation) as a function of P_{C}. FIGS. 7A and 7B present two examples of the experimental TD vs. P_{C} curve (above) and the corresponding ΔTD vs. P_{C} curve. As can be seen, these best-fit approximations generally generate only one big wave, which can easily be identified. The value of P_{C}, which is associated with the pulse of maximum value of ΔTD, was taken as DBP, with no need for the first stage of rough approximation of DBP.

In a copending patent application (U.S. patent application Ser. No. 09/545,190, hereby incorporated by reference), we suggested applying relatively low air pressure, of about 40 mmHg, on the arm by the pressure cuff for a short time, say 10 sec, before the start of the rapid inflation. This preceding pressure was suggested for avoiding the closure of the arteries by the cuff and drainage of their blood into the veins during the rapid inflation, resulting in collapse of the arteries under the PPG probe. This closure, if allowed to occur, reduces the accuracy in the measurement of SBP by the PPG method. The preceding pressure closes the veins under the cuff, but not the arteries, so that blood can flow into the hand and accumulate in the veins, preventing significant drainage of the arteries.

A similar procedure, i.e. prior application of low cuff pressure, can also be used according to the teachings of this invention to increase the accuracy of DBP measurement. The dependence of TD on P_{C} was found to change between examinations performed on the same person, probably due to natural differences in venous blood content. The ΔTD vs. P_{C} curve (which is obtained during the deflation period) was found to be more reproducible if the rapid inflation of the cuff pressure to above SBP value was preceded by applying low air pressure, of about 40 mmHg, on the arm for a short time, in a manner similar to that described in the aforementioned U.S. application.

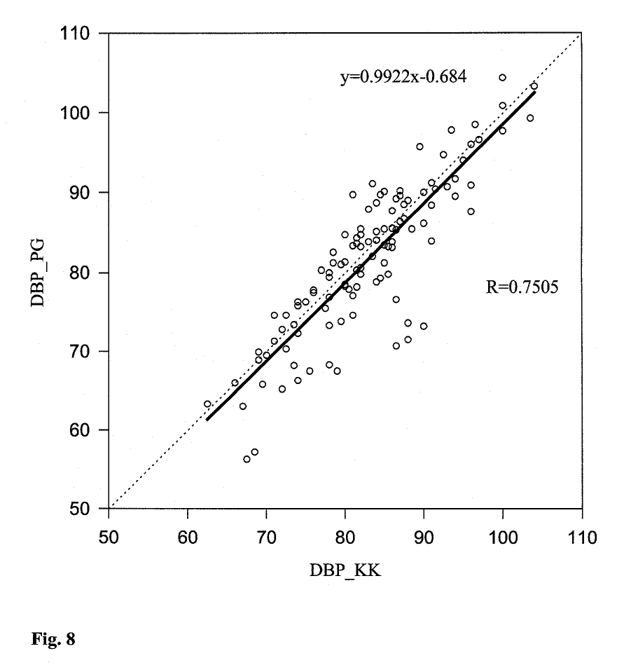

FIG. 8 shows the value of DBP obtained by the PPG method, DBP_{P} as a function of the value of DBP obtained by manual SPG, DBP_{S}. The standard deviation of the values of the difference between the two methods is 4.8 mmHg, significantly lower than maximum 8 mmHg required by BHS. This value of SD is similar to that claimed in our U.S. patent, but the results in the former U.S. patent were obtained from measurements on healthy persons, while the data on the current patent application were obtained from measurements on heterogeneous group which included elderly persons and hypertensive and diabetic patients as well.

It will be appreciated that the above descriptions are intended only to serve as examples, and that many other embodiments are possible within the scope of the present invention as defined by the appended claims.

What is claimed is:

1. A method for measuring arterial diastolic blood pressure in a subject, the method comprising:
(a) generating first and second signals indicative, respectively, of cardiac induced pulsatile variations of a cardiovascular parameter in a first region and a second region of the subject's body;
(b) processing said first and second signals to derive values of a delay between pulses in said first signal and corresponding pulses in said second signal;
(c) applying a variable pressure to a pressure application region of the subject's body so as to affect blood flow through at least one artery in said pressure application region, said variable pressure being varied over time, said first region, said second region and said pressure application region being chosen such that said delay varies as a result of changes in said variable pressure;
(d) deriving parameters of a mathematical function such that said function corresponds approximately to a relationship between said delay and said variable pressure, said mathematical function being monotonic;
(e) calculating a difference between said values of said delay and corresponding values on said mathematical function; and
(f) identifying as the diastolic blood pressure a value of the variable pressure for which said difference exhibits a stationary point.

2. The method of claim 1, wherein said mathematical function is an exponential function.

3. The method of claim 1, wherein said mathematical function is a power-curve function.

4. The method of claim 1, wherein said mathematical function is a polynomial function.

5. The method of claim 1, further comprising, prior to said step of identifying, obtaining an approximate value of the diastolic blood pressure, and wherein said identifying includes selecting a stationary point of said difference from a plurality of stationary points by proximity to said approximate value.

6. The method of claim 5, wherein said approximate value is obtained by identifying a value of said applied pressure at which said delay assumes a predefined non-zero value.

7. The method of claim 6, wherein said predefined non-zero value of said delay is calculated from a predefined function of the systolic blood pressure of the subject.

8. The method of claim 1, wherein said parameters are derived according to a best-fit criterion.

9. The method of claim 1, wherein said parameters are derived according to a least-squares best-fit criterion.

10. The method of claim 1, wherein both said first and said second signals are generated by PPG sensors.

11. The method of claim 1, wherein at least one of said first and said second signals is generated by a PPG sensor.

12. The method of claim 11, wherein one of said first and said second signals corresponds to oscillations in air pressure within a cuff used to apply said variable pressure.
